(19) United States
(12) Patent Application Publication
(10) Pub. No.: US 2006/0210659 A1
(43) Pub. Date: Sep. 21, 2006

(54) Anti-obesity agent

(75) Inventors: Isao Nadaoka, Ibaraki (JP); Hiroshi Sugiyama, Ibaraki (JP)

Correspondence Address: HOFFMANN & BARON, LLP, 6900 JERICHO TURNPIKE, SYOSSET, NY, 11791, US

(73) Assignee: Asahi Breweries, Ltd.

(21) Appl. No.: 11/377,795

(22) Filed: Mar. 16, 2006

(30) Foreign Application Priority Data
Mar. 16, 2005 (JP) 2005-075610

(51) Int. Cl. A61K 36/71; A61K 31/704; A61K 31/20

(52) U.S. Cl. 424/774; 514/33; 514/559

(57) ABSTRACT

The object of the present invention is to provide an active substance which has an anti-obesity effect such as a triglyceride lowering activity, a cholesterol lowering activity and the like. The present invention provides a blood triglyceride lowering agent, a cholesterol lowering agent, a body fat storage suppressive agent, an anti-obesity agent and an antilipemic agent, characterized in that each of said agents comprises a cycloartane-type triterpene or glycoside thereof. Moreover, the present invention provides the use of a Black cohosh plant in order to prepare said agents. Further, the present invention provides a beverage, food and quasi-drug comprising said agents.

### TECHNICAL FIELD

The present invention relates to a blood triglyceride lowering agent, a cholesterol lowering agent, a body fat storage suppressive agent, an anti-obesity agent and an antilipemic agent, characterized in that each of said agents comprises a cycloartane-type triterpene or glycoside thereof. The cycloartane-type triterpene or glycoside thereof used as an active substance in the present invention can be obtained from, for example, a Black cohosh plant.

### BACKGROUND ART

The tendency for the people to become obesity and hypertension due to high fat and high calorie diet has been gradually proceeding. Obesity in the upper half of the body, disorders of carbohydrate metabolism, high triglyceride level and hypertension are referred to as “quartet of death”, so these conditions coincide one another and then the morbidity of arterioscleroses or cardiac diseases increases acceleratedly. The expression of such physiological abnormalities is largely involved in diet and other environment factors. It is necessary to do change the lifestyle in order to prevent and improve such conditions, so voluntary control which is difficult to be continued may be forced. It is desirable to find an easy method for preventing and improving arterioscleroses, which can be acceptable by anyone, such as ingestion of functional foods.

In recent years, a variety of useful substances have been found from foods or natural products corresponding to them, and then functional foods which make use of their physiological functions and safety are coming to boom.

Black cohosh is a vegetation of Cimicifuga in Ranunculaceae, whose botanical name is Cimicifuga racemosa. Cimicifuga racemosa have been eaten by Indians in the north America for the purpose of analgesic or improving climacteric disorder for a long time, and they have been still distributed as an edible herb in Europe and America. Cimicifugine which is one of components of Cimicifuga racemosa is known to suppress the rapid decrease of estrogen to alleviate the symptoms of climacteric disorder. The use of Cimicifuga racemosa extracts has been disclosed as estrogen-type period selective agent in order to treat and/or prevent cardiovascular disease, atherosclerosis, osteoporosis and climacteric disorder, for example, in order to prevent or alleviate transitory hot flushes (see, for example, JP2002506827A). Anti-obesity effects such as triglyceride lowering activity and cholesterol lowering activity are, however, not mentioned in it.

### DISCLOSURE OF THE INVENTION

In order to prevent and/or treat obesity which would threaten human health for a long time, it is necessary to find active substances from foods and those which correspond to them.

The present inventors have made various studies to solve the above problems and have consequently found that the cycloartane-type triterpene and glycoside thereof contained in a Black cohosh plant is effective in preventing obesity and the like, and then they have accomplished the present invention.

That is to say, the present invention provides a blood triglyceride lowering agent, characterized in that said agent comprises a cycloartane-type triterpene or glycoside thereof.

Moreover, the present invention provides a cholesterol lowering agent, characterized in that said agent comprises a cycloartane-type triterpene or glycoside thereof.

Further, the present invention provides a body fat storage suppressive agent, characterized in that said agent comprises a cycloartane-type triterpene or glycoside thereof.

In addition, the present invention provides an anti-obesity agent, characterized in that said agent comprises a cycloartane-type triterpene or glycoside thereof.

Further, the present invention provides an antilipemic agent, characterized in that said agent comprises a cycloartane-type triterpene or glycoside thereof.

Moreover, the present invention provides the use of a Black cohosh plant in order to prepare said agents.

Further, the present invention provides a beverage, food and quasi-drug comprising said agents.

### EFFECT OF THE INVENTION

The pharmaceutical as well as the food and beverage according to the present invention have an anti-obesity activity and then it is effective in preventing and treating lifestyle diseases.

### BEST MODE FOR CARRYING OUT THE INVENTION

The cycloartane-type triterpene and glycoside thereof used in the present invention include, but are not limited to, for example, actein, 23-epi-26-deoxyactein, cimigenole 3-0-α-L-arabinopyranoside, cimigenole 3-0-β-D-xylopyranoside, 25-0-acetylcimigenole 3-0-α-L-arabinopyranoside, 25-0-acetylcimigenole 3-0-β-D-xylopyranoside, 12-hydroxycimigenole 3-0-α-L-arabinopyranoside cimiracemoside F, 25-0-methoxy cimigenole 3-0-α-L-arabinopyranoside, 12,21-dihydroxycimigenole 3-0-α-L-arabinopyranoside and derivatives thereof and the like. Preferably it is actein or derivatives thereof.

The cycloartane-type triterpene and glycoside thereof used in the present invention is contained in plant bodies of Cimicifuga simplex genus, Cimicifuga japanica genus, Cimicifuga acerina genus, Cimicifuga dahurica genus, Cimicifuga heracleifolia genus, Cimicifuga foetida genus in Ranunculaceae, such as a Black cohosh and the like. When a blood triglyceride lowering agent, a cholesterol lowering agent, a body fat storage suppressive agent, an anti-obesity agent and an antilipemic agent according to the present invention are prepared, these plant bodies may be used as they are, or extracts from these plant bodies may be used as active substances. For example, when a Black cohosh plant is used as it is, leaves, fruits, seeds, trunks or roots, which are in raw states or in the states after drying, are ground into appropriate sizes or powdered. When the extract is used as an active substance, water, alcohol, other organic solvents or the like are used as extractive solvents. The mixture of these solvents may be also used. Preferred extractive solvent is water, or a mixture of water and alcohol, etc. The extraction may be performed by room temperature extraction, hot extraction, further pressure extraction or the like. Generally, the extraction is carried out at room temperature to 125° C. After extracting the extracts from plant bodies, solid contents and liquids are separated by centrifugation or the like and another treatment such as filtration may be performed, as necessary, followed by vacuum concentration or the like to concentrate. In addition, the extracts can be also powdered by vacuum drying, lyophilization and the like. During powdering, an appropriate excipient may be added. Alternatively, the extracts may be further purified to isolate cycloartane-type triterpene such as actein or glycoside thereof, and then used to prepare the blood triglyceride lowering agent, cholesterol lowering agent, body fat storage suppressive agent, anti-obesity agent and antilipemic agent according to the present invention.

When the blood triglyceride lowering agent, cholesterol lowering agent, body fat storage suppressive agent, anti-obesity agent and antilipemic agent according to the present are prepared, the conventional method of formulation may be suitably used, so auxiliary agents conventionally used such as an excipient, binder, lubricant, disintegrator, flavor improvement agent, dissolution-aid, suspending agent, coating agent and the like may be added to a plant containing cycloartane-type triterpene or glycoside thereof or extracts therefrom as well as a simple substance of cycloartane-type triterpene or glycoside thereof to formulate in the form of tablets, powders, capsules, beverages or the like.

Moreover, an excipient alone or together with food materials may be added to a plant containing cycloartane-type triterpene or glycoside thereof or extracts therefrom as well as a simple substance of cycloartane-type triterpene or glycoside thereof to formulate into solid form (powder, granular or the like), paste form, liquid form or suspended form, or to make the form of beverage or food, for example, dairy products such as fermented milk, cheese, butter and the like, drink yogurt or lactic acid bacteria beverage, confectionery and baked goods such as butter cake, or further to make into quasi-drugs such as a supplement. Food materials may be anyone of solid materials (powdery, flaky, massive or the like), semi-solid materials (jellied, thick malt syrup-like or the like), or liquid materials and the like. It is desirable that the content of cycloartane-type triterpene or glycoside thereof is from 0.1 to 5.0 mg (dry weight) per gram of the pharmaceuticals, beverages, foods or quasi-drugs.

Because the cycloartane-type triterpene or glycoside thereof used in the present invention is no problem in safety, there is no limitation of the dose in the case of orally administrating it. Generally, a dose used for foods is set, specifically the amount from 0.1 to 25 mg (dry weight)/kg of body weight, preferably from 0.1 to 5.0 mg (dry weight)/kg of body weight is administrated one or more times per day. Moreover, the daily total dose is from 0.1 to 25 mg, preferably 0.1 to 5.0 mg.

### EXAMPLES

### Example 1

### The Purification of Black Cohosh Extract

Black cohosh rhizome (5.2 kg) was extracted by 10 L of 50% hydrate ethanol, concentrated under vacuum, and then dried to obtain 445 g of the extract. The content of cycloartan-type triterpene or glycoside thereof in said extract was 11 g.

### Example 2

### The Extraction, Purification and Isolation of Actein

Black cohosh rhizome (5.2 kg) was extracted by hot methanol (3 hours, 2 times), the extract was concentrated under vacuum. 445 g of the concentrated extract was suspended in 30% methanol, added to Diaion HP-20 column chromatography, and eluted by 30% methanol, 50% methanol, methanol, ethanol and ethyl acetate, while the polarity was decreased in turn, to fractionate to five crude fractions. 181 g of the resulting methanol eluting fraction was fractionated to seven fractions (fractions A-G) by silica gel column chromatography [chloroform-methanol (19:1, 9:1, 4:1, 2:1) methanol]. Fraction C was fractionated to further five fractions (fractions C-1 to C-5) by silica gel column chromatography [chloroform-methanol (19:1)], octadecylsilanized (ODS) silica gel column chromatography [acetonitrile-water (1:1)], Sephadex LH-20 [methanol]. Fraction C-3 was dissolved in methanol, allowed to stand at room temperature, and then precipitate was produced. The precipitate was collected by filtration and purified by repeating silica gel column chromatography [chloroform-methanol (30:1, 19:1)], ODS silica gel column chromatography [acetonitrile-water (1:1)] and semi-fractional ODSHPLC [acetonitrile-methanol-water (1:1:1)] to isolate actein (3.2 g) of the following structure formula.

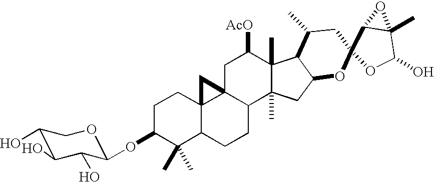

### Example 3

### The Lipid-Absorption Suppressive Activity of Black Cohosh Extract

Orbital blood collection from male ddy line mice of six weeks old (six animals per group) was carried out, and then 10 mL/kg of water or 0.1 g/kg of aqueous actein solution was orally administrated to each of them. After 30 minutes, 10 mL/kg of salad oil was orally administrated, orbital blood collection was performed after 180 minutes again, and blood plasma was separated to measure the triglyceride level. Due to the administration of actein, the triglyceride level in blood plasma was significantly suppressed after 180 minutes of salad oil tolerance (see Table 1).

TABLE 1
The triglyceride level in blood plasma of each test
group (mean value ± standard deviation)

| | Triglyceride level in blood plasma (mg/dL) | |
|---|---|---|
| Administrated liquid | Before salad oil After 180 minutes administration | |
| Water | 132.7 ± 14.3 | 448.1 ± 49.0 |
| 0.1 g/kg actein | 110.4 ± 22.2 | 248.6 ± 27.8 ** |

(** p < 0.01 vs water administration group)

### Example 4

### The Lipid-Absorption Suppressive Activity of Black Cohosh Extract

Orbital blood collection from male ddy line mice of six weeks old (six animals per group) was carried out, and then 10 mL/kg of water or 0.2 g/kg or 1.0 g/kg of aqueous solution of Black cohosh extract (Example 1) was orally administrated. After 30 minutes, 10 mL/kg of salad oil was orally administrated, orbital blood collection was performed after 180 minutes again, and blood plasma was separated to measure the triglyceride level. Due to the administration of Black cohosh, the triglyceride level in blood plasma was significantly suppressed after 180 minutes of salad oil tolerance (see Table 2).

TABLE 2
The triglyceride level in blood plasma of each test
group (mean value ± standard deviation)

| | Triglyceride level in blood plasma (mg/dL) | |
|---|---|---|
| Administrated liquid | Before salad oil After 180 minutes administration | |
| Water | 135.2 ± 18.7 | 317.4 ± 45.0 |
| 0.2 g/kg Black cohosh | 105.5 ± 11.3 | 139.9 ± 23.1 ** |
| 1.0 g/kg Black cohosh | 92.9 ± 12.7 | 145.9 ± 34.3 ** |

(** p < 0.01 vs water administration group)

### Example 5

### The Anti-Obesity Activity of Black Cohosh Extract

Each ten male CBA/N line mice of eight weeks old were separated into two groups, each of them was allowed to take freely purified feed (CRF-1) or purified feed containing 0.5% of Black cohosh extract (Example 1) powder for four weeks. After the completion of breeding, the measurement of animals' weight and whole blood collection were performed, blood plasma was separated to measure the triglyceride level, total cholesterol level and blood sugar level. Moreover, the lipid around testicle was ablated to weigh.

As shown in Table 3, it was confirmed that the lipid around testicle and body weight were decreased in the group fed purified feed containing Black cohosh extract.

TABLE 3
The physique of the animals in each test group
(mean value ± standard deviation)

| | Body weight | Lipid around |
|---|---|---|
| Test group | (g) | testicle (g) |
| Usual purified feed | 30.2 ± 0.5 | 3.17 ± 0.14 |
| 0.5% Black cohosh addition | 27.1 ± 0.5** | 2.44 ± 0.20* |

(**p < 0.01,
*p < 0.05 vs usual purified feed group)

As further shown in Table 4, any of the triglyceride levels and total cholesterol levels of the group fed purified feed containing Black cohosh extract were lower than those of the group fed purified feed only, with the proviso that blood sugar levels were not changed.

TABLE 4
The blood plasma biochemical values (mean
value ± standard deviation)

| | Triglyceride | Total cholesterol |
|---|---|---|
| Test group | level (mg/dL) | level (mg/dL) |
| Usual purified feed | 381.0 ± 25.8 | 150.3 ± 8.4 |
| 0.5% Black cohosh addition | 276.2 ± 20.0* | 127.2 ± 1.8* |

(*p < 0.01 vs usual purified feed group)

### Example 6

### Tablets and Capsules

| | |
|---|---|
| Black cohosh extract (Example 1) powder | 50.0 g |
| Lactose | 35.0 g |
| Magnesium stearate | 15.0 g |
| Total | 100.0 g |

The above each weight part of components were uniformly mixed to provide tablets and capsules according to conventional methods.

### Example 7

### Powders and Granules

| | |
|---|---|
| Black cohosh extract (Example 1) powder | 10.0 g |
| Starch | 40.0 g |
| Lactose | 50.0 g |
| Total | 100.0 g |

The above each weight part of components were uniformly mixed to provide powders and granules according to conventional methods.

### Example 8

### Candies

| | |
|---|---|
| Black cohosh extract (Example 1) powder | 10.0 g |
| Thick malt syrup (75% by weight of solid content) | 80.0 g |
| Water | 9.5 g |
| Coloring agent (Daiwa Kasei Co., Ltd. | 0.45 g |
| High Orange W-150) | |
| Flavor (T. Hasegawa Co., Ltd. | 0.05 g |
| Orange Flavor AH-11444) | |
| Total | 100.0 g |

The above each weight part of components were used to provide candies according to conventional methods.

### Example 9

### Troches

| | |
|---|---|
| Gum arabic | 6.0 g |
| Magnesium stearate | 3.0 g |
| Glucose | 63.0 g |
| Lactose | 17.6 g |
| Potassium secondary phosphate | 0.2 g |
| Potassium primary phosphate | 0.1 g |
| Flavor (T. Hasegawa Co., Ltd. Orange Flavor) | 0.1 g |
| Black cohosh extract (Example 1) powder | 10.0 g |
| Total | 100.0 g |

The above each weight part of components were used to provide troches according to conventional methods.

### Example 10

### Juice

| | |
|---|---|
| Concentrated orange juice | 15.0 g |
| Fructose | 5.0 g |
| Citric acid | 0.2 g |
| Flavor (Takasago International Corporation, | 0.1 g |
| Orange Flavor) | |
| Coloring matter (San-Ei Gen F.F.I, Inc. | 0.15 g |
| Orange color base) | |
| Sodium ascorbate | 0.05 g |
| Black cohosh extract (Example 1) powder | 0.1 g |
| Water | 79.4 g |
| Total | 100.0 g |

The above each weight part of components were used to provide juice according to conventional methods.

### Example 11

### Cookies

| | |
|---|---|
| Soft flour | 32.0 g |
| Whole egg | 16.0 g |
| Butter | 16.0 g |
| Sugar | 25.0 g |
| Water | 10.6 g |
| Baking powder | 0.2 g |
| Black cohosh extract (Example 1) powder | 0.2 g |
| Total | 100.0 g |

The above each weight part of components were used to provide cookies according to conventional methods.

### Example 12

### Chewing Gums

| | |
|---|---|
| Gum base | 20.0 g |
| Calcium carbonate | 2.0 g |
| Lactose | 73.0 g |
| Stevioside | 0.1 g |
| Flavor (T. Hasegawa Co., Ltd. Orange Flavor ) | 0.9 g |
| Black cohosh extract (Example 1) powder | 4.0 g |
| Total | 100.0 g |

The above each weight part of components were used to provide chewing gums according to conventional methods.

### Example 13

### Infusion of Parched Barley

| | |
|---|---|
| Parched barley | 5.0 g |
| Water | 2995 g |
| Total | 3000 g |

The above each weight part of components were used to extract parched barley component according to conventional methods, and then the following components were combined with it to provide infusion of parched barley.

| | |
|---|---|
| Sodium ascorbate | 1.8 g |
| Black cohosh extract (Example 1) powder | 1.2 g |

### Example 14

### Infusion of Green Tea

| | |
|---|---|
| Green tea | 5.0 g |
| Water | 2995 g |
| Total | 3000 g |

The above each weight part of components were used to extract green tea component according to conventional methods, and then the following components were combined with it to provide infusion of green tea.

| | |
|---|---|
| Sodium ascorbate | 1.8 g |
| Black cohosh extract (Example 1) powder | 1.2 g |

### Example 15

### Coffee beverage

| | |
|---|---|
| Coffee extract | 28.0 g |
| Milk | 10.0 g |
| Nonfat powdered milk | 0.5 g |
| Sugar | 5.0 g |
| Emulsifying agent | 0.14 g |
| Sodium bicarbonate | adjusts to pH 6.8 |
| Black cohosh extract (Example 1) powder | 0.1 g |
| Water | q.s. |
| Total | 100.0 g |

The above each weight part of components were used to provide coffee beverage according to conventional methods.

### Example 16

### Low-Alcoholic Beverage

| | |
|---|---|
| 95 v/v % alcohol | 76.0 ml |
| Fructose glucose liquid sugar | 30.0 g |
| Fruit juice | 5.0 g |
| Citric acid | 0.5 g |
| Sodium ascorbate | 0.3 g |
| Black cohosh extract (Example 1) powder | 0.6 g |
| Carbonated water | q.s. |
| Total | 1000 g |

The above each weight part of components were used to provide low-alcoholic beverage according to conventional methods.

### Example 17

### Tea Bag Beverage

5.0 g of Black cohosh extract (Example 1) powder was packed in a bag to provide a tea bag.

1. A method for lowering blood triglyceride, characterized in that said method comprises administering a cycloartane-type triterpene or glycoside thereof.

2. A method for lowering cholesterol, characterized in that said method comprises administering a cycloartane-type triterpene or glycoside thereof.

3. A method for suppressing body fat storage, characterized in that said method comprises administering a cycloartane-type triterpene or glycoside thereof.

4. A method for suppressing obesity, characterized in that said method comprises administering a cycloartane-type triterpene or glycoside thereof.

5. A method for suppressing hyperlipidemia, characterized in that said method comprises administering a cycloartane-type triterpene or glycoside thereof.

6. The method according to claim 1, wherein said cycloartane-type triterpene or glycoside thereof is an actein or derivative thereof.

7. The method according to claim 2, wherein said cycloartane-type triterpene or glycoside thereof is an actein or derivative thereof.

8. The method according to claim 4, wherein said cycloartane-type triterpene or glycoside thereof is an actein or derivative thereof.

9. The method according to claim 1, wherein said agent comprises a plant of Cimicifuga simplex genus, Cimicifuga japanica genus, Cimicifuga acerina genus, Cimicifuga dahurica genus, Cimicifuga heracleifolia genus or Cimicifuga foetida genus in Ranunculaceae.

10. The method according to claim 2, wherein said agent comprises a plant of Cimicifuga simplex genus, Cimicifuga japanica genus, Cimicifuga acerina genus, Cimicifuga dahurica genus, Cimicifuga heracleifolia genus or Cimicifuga foetida genus in Ranunculaceae.

11. The method according to claim 4, wherein said agent comprises a plant of Cimicifuga simplex genus, Cimicifuga japanica genus, Cimicifuga acerina genus, Cimicifuga dahurica genus, Cimicifuga heracleifolia genus or Cimicifuga foetida genus in Ranunculaceae.

12. The method according to claim 9, wherein said plant of Cimicifuga in Ranunculaceae is a Black cohosh.

13. The method according to claim 10, wherein said plant of Cimicifuga in Ranunculaceae is a Black cohosh.

14. The method according to claim 11, wherein said plant of Cimicifuga in Ranunculaceae is a Black cohosh.

15. Use of a Black cohosh plant in order to prepare an agent selected from the group consisting of a blood triglyceride lowering agent, a cholesterol lowering agent, a body fat storage suppressive agent, an anti-obesity agent and an antilipemic agent.

16. Use of a Black cohosh plant in order to prepare a beverage, food or quasi-drug comprising an agent selected from the group consisting of a blood triglyceride lowering agent, a cholesterol lowering agent, a body fat storage suppressive agent, an anti-obesity agent and an antilipemic agent.
